(19) United States
(12) Patent Application Publication
(10) Pub. No.: US 2002/0022215 A1
(43) Pub. Date: Feb. 21, 2002

(54) Inactivation of small non-enveloped viruses and other microbial pathogens by porphyrins

(76) Inventors: Mark D. Sobsey, Chapel Hill, NC (US); Avram Gold, Chapel Hill, NC (US); Michael J. Casteel, Durham, NC (US)

Correspondence Address: MYERS BIGEL SIBLEY & SAJOVEC, PO BOX 37428, RALEIGH, NC, 27627, US

(21) Appl. No.: 09/895,864

(22) Filed: Jun. 29, 2001

Related U.S. Application Data
(60) Provisional application No. 60/214,954, filed on Jun. 29, 2000.

(51) Int. Cl. A01N 1/02; C12Q 1/22

(52) U.S. Cl. 435/2; 435/31

(57) ABSTRACT

Microbial pathogens present in a fluid medium may be inactivated by contacting the medium with a light-activated porphyrin and then irradiating the medium with light (e.g., ultraviolet light). Blood products (e.g., whole blood, plasma) are examples of fluid media that may be treated in this manner.

### RELATED APPLICATIONS

This application claims the benefit of U.S. Provisional Application No. 60/214,954, filed Jun. 29, 2001, which is incorporated herein by reference in its entirety.

### FIELD OF THE INVENTION

The present invention relates to methods of using porphyrins to inactivate viruses and microbial pathogens.

### BACKGROUND OF THE INVENTION

Millions of units of blood components are transfused annually worldwide, under conditions ranging from sophisticated to primitive. The risk of transmitting infections such as HIV, hepatitis B (HBV), and hepatitis C (HCV) has been substantially reduced by donor testing and exclusion, and also by implementation of inactivation procedures such as heat- and/or solvent-detergent-treatment of plasma. However, such treatments may impair protein functions or the integrity of cellular components. Furthermore, detergent treatment is ineffective against non-enveloped viruses. Donor screening does not provide absolute safety, nor does increasingly sophisticated testing protect against newly emerging blood-borne pathogens. Alternative methods for inactivation of pathogens in blood and its components are needed.

Of particular concern are the small, non-enveloped viruses such as Parvovirus B19 (PVB19) and hepatitis A virus (HAV). These are small (23-27 nanometers in diameter), non-enveloped human pathogens that contain ribonucleic acid (e.g., RNA in HAV) or deoxyribonucleic (e.g., DNA in PVB19) genomes. These viruses have been identified in numerous instances of blood-borne transfusion disease transmission. Factor VII concentrates and immunoglobulins have been implicated as the source of infection of infectious hepatitis caused by HAV and the disease(s) associated with PVB19 (Erdmann et al., 1997. J Med Virol, 53, 233-236). Viruses such as HAV and PVB19 have been demonstrated to be resistant to the removal and inactivation procedures that show efficacy against lipid-enveloped viruses such as HIV, HBV, and HCV.

Bacterial contamination of blood products is also of ongoing concern. One in 1000 to one in 2000 platelet concentrates are estimated to be contaminated (Dykstra et al., 1998. Transfusion 38, 104S), and approximately 150 cases of severe morbidity, including many fatalities, are reported annually in the U.S.A. alone (Amer. Association of Blood Banks, 1996. Association Bulletin #96-6 Bacterial Contamination of Blood Components. AABB Faxnet, No. 294). Under-reporting and under-recognition of contamination imply that these numbers may be higher. The organisms most commonly detected are include Staphylococcus epidermidis and Bacillus sp., and include both gram-positive and gram-negative bacteria (Mitchell and Brecher, 1999. Transfusion Medicine Reviews, 13(2),132-144).

Photochemical processes (Ben-Hur and Horowitz, 1995. Photochem Photobiol 62, 383-388) for inactivating microbes in blood products are known. Treatment with UVC light was found to be effective against non-enveloped viruses in plasma (Chin et al., 1995. Photochem Photobiol, 65(3), 432-435). However, the potential carcinogenicity of UVC and other UV light wavelength ranges requires caution when irradiating whole blood or leucocyte preparations. Accordingly, light alone is as a treatment not a desirable option.

Use of photoactive compounds, called photosensitizers, enables chemical enhancement of the light energy. On exposure to light of an appropriate wavelength, a photosensitizer absorbs energy and is converted from the stable electronic ground state S₀ to a short-lived, excited singlet state S₁*. The latter may drop back to the ground state with emission of light (as fluorescence), heat, or chemical reaction with another molecule. Alternatively, the excited singlet may convert to a longer-lived metastable triplet state. Direct production of a free radical from reaction of an electron or hydrogen donor with the excited sensitizer can result in a Type I reaction (direct toxicity) with adjacent molecules, biomolecules or the sensitizer itself. A Type II reaction occurs under aerobic conditions, when the metastable triplet transfers energy to molecular oxygen, to give the excited state singlet oxygen. Singlet oxygen itself is highly reactive towards biological macromolecules, and can initiate sequences of radical-mediated reactions such as lipid peroxidation. In addition, the photosensitizer ultimately reverts to its ground state, and can repeat the process given a sufficient supply of oxygen.

Several photosensitizers have been proposed for disinfection of blood products, and some are currently in clinical trials. The phenothiazine dye methylene blue was shown to inactivate enveloped viruses (including HSV and HIV1) in plasma, but was less effective against non-enveloped viruses (Lambrecht et al., 1991. Vox Sang. 60(4), 207-13). Methylene blue gives rise to photoproducts that are themselves photoactive. Development of this compound has been actively pursued (Hirayama et al., 2000. Photochemistry and Photobiology, 71(1), 90-93), and has reached the stage of clinical trials in Europe (Simonsen and Sorensen, 1999. Vox Sang, 77, 210-217). Enveloped viruses including HSV1, HIV and CMV were inactivated by the negatively-charged polymethine dye Merocyanine 540 (Sieber et al., 1992. Blood Cells, 18, 117-128) and white light. Although this agent has the advantage over many photosensitizers in that it does not accumulate in the skin and does not damage red cells. Moreover, it is toxic to white blood cells and causes loss of platelets in platelet concentrates.

The polycyclic dianthraquinone hypericin, which absorbs light at longer wavelengths, was effective found to be against enveloped viruses in red blood cell concentrates. However, hypericin must be complexed with albumin in order to decrease phototoxic hemolysis to acceptable levels, which decreases disinfection efficiency (Prince et al., 2000. Photochemistry and Photobiology, 71(2), 188-195).

Psoralens, being prototypical photosensitizers, have also been evaluated as disinfectants. They are known to intercalate into DNA and form cross-links; hence a mechanism for their mode of action can readily be postulated. By the same reasoning, this class of compounds can also be predicted to pose a genotoxic risk, and should be avoided if effective alternatives exist. 8-MOP (8-methoxypsoralen) and PUVA (psoralens plus UVA light) are classified as human carcinogens by both the U.S. Public Health Service (US PHS) and by the International Agency for Research on Cancer (IARC, 1987).

Several phthalocyanine dyes are active against enveloped viruses (Zmudka et al., 1997. Photochem. Photobiol., 65(3), 461-464; Smetana et al., 1998. J. Photchem. Photobiol. B.: Biol., 44(1), 77-83), and a zinc phthalocyanine complex has been reported to inactivate both gram-positive and gram-positive bacteria (Minnock et al., 1996. J Photochem Photobiol B: Biol, 32,159-164). While phthalocyanines have the advantage of high absorbance at the red end of the visible spectrum, synthetic routes to these compounds are limited and yield mixtures of products that are difficult to purify (Bonnett, 1995. Chemical Society Reviews, 19-33). Agents that are effective against non-enveloped viruses and are also compatible with blood and blood products have not heretofore been described.

General difficulties associated with potential photoactivated disinfectants can be attributed to the wavelength of the activating light. UV radiation can be genotoxic and carcinogenic, while visible light is absorbed by the medium. The latter is particularly a problem with packed red blood cells, in which the intense absorbance of hemoglobin (up to approximately 640 nm) dictates that a photosensitizer be sought with one or more strongly light-absorbing wavelengths above this band. In specific cases such as methylene blue, the agent forms photo-products, which are themselves active and exhibit toxicity. It has been suggested that the effectiveness of inactivation dependent on the Type II reaction is decreased in platelet concentrates (Santus et al., 1998. Clin Hemor Microcirc, 18, 299-308). Moreover, inactivation of intra-cellular viruses without causing general loss of cellular integrity is of particular concern with pathogens such as HIV. The chemical agent may bind to plasma proteins (especially non-specific binding to albumin), leading to decreased activity and potential antigenicity. The agent may inactivate clotting factors or enzymes, and may cause damage to blood cells. These possibilities are of concern for any photosensitizer to be used with blood products. In addition, where the agent remains in the product during transfusion, the risk of post-transfusion photosensitivity exists until the agent has been eliminated from the body.

Porphyrins are tetrapyrrolic compounds that containing the porphine structure of four pyrrole rings connected by methine bridges in a cyclic configuration, to which a variety of side chains are attached. Porphyrins in general have the following basic skeletal structure and numbering convention.

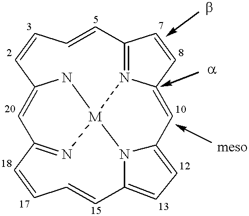

Two substituent patterns are defined: (i) a physiological substituent pattern in which the pyrrole β-positions (C2,C3,C7,C8,C12,C13,C17,C18) bear alkyl groups and (ii) a non-physiological substituent pattern in which the meso carbons (C5,C10,C15,C20) bear aryl or alkyl substituents (e.g., is “meso-substituted”). Porphyrins complexed with transition metals, having partially-filled 3d atomic orbitals, are ineffective as photosensitizers because they are auto-quenching. Metal-porphyrin complexes may also be more prone to photodegradation (Fuhrhop, 1974. Agnew. Chem. Internat 13(5), 321-335) and metabolism by heme oxidase (Ortiz de Montellano, 1998. Acc. Chem. Res., 31, 543-549) than are the free bases, and may therefore offer more possibilities for toxic breakdown products. Both physiologically-substituted and non-physiologically-substituted types of porphyrin have demonstrated high quantum yields of singlet oxygen and are resistant to physical degradation (Bonnett, 1995. Chemical Society Reviews, 19-33; Jori and Reddi, 1991. Light in Biology and Medicine, 2, 253-266; Merchat et al., 1996. J Photochem Photobiol B: Biol, 35(3), 149-57; Verlhac et al., 1994. Nouv. J. Chim., 8, 401-406). Based on toxicological and mutagenicity assessments carried out for use of porphyrins in photodynamic cancer therapy clinical trials, porphyrins as a class would be expected to exhibit low toxicity.

Several porphyrins have been tested in clinical trials for a number of medical uses (Bonnett, 1995. Chemical Society Reviews, 19-33; Kreimer-Birnbaum, 1989. Seminars in Hematology, 26(2),157-173). These include porphyrins or porphyrin derivatives with physiological substituent patterns, such as Photofrin™ and hematoporphyrin, and compounds with non-physiological substituent patterns, such as the dihydro tetrakis(m-hydroxyphenyl) porphyrin Temoporphin™ (Bonnett, 1995. Chemical Society Reviews, 19-33). More recent efforts to develop porphyrins for therapeutic uses have favored the non-physiological substituent pattern because of easier synthetic availability and the concomitant convenience of obtaining single compounds in high purity.

One attractive feature of porphyrins is the variety of functional groups that can be formed upon substitution to the base structure of the porphyrin molecule. This feature allows conferring changes in overall charge, light absorption, hydrophobicity and hydrophilicity, and propensity for Type I or Type II photosensitization. In addition, porphyrins can interact with DNA or RNA via a variety of different mechanisms (Di Mauro et al., 1998. J Molecular Biol 282, 43-57). Porphyrins may be added directly into tubing or bags, or immobilized onto microbeads, resins, membranes, or other solid-phase formats. Porphyrins have also been conjugated with monoclonal antibodies, or to nucleic acid probes for specific adsorption to target nucleic acids or certain tissues or cell types (Benimetskaya et al., 1998. Nucleic Acids Research 26(23), 5310-7; Flynn et al., 1999. BioTechniques 26, 736-746). In addition, porphyrin compounds have been shown to inhibit accumulation of the protease-resistant proteins (prions) that cause transmissible encephalopathy in animals and humans (Priola et al., 2000. Science, 287, 1503-1506; Caughey et al., 1998. Proc Natl Acad Sci USA, 95, 12117-12122). However, practical applications of porphyrins outside PDT cancer therapy are not as yet extensively documented.

Activity of porphyrins against bacteria has been demonstrated in vitro (Valduga et al., 1999. Biochem Biophys Res Comm 256(1):84-88; Merchat et al., 1996. J Photochem Photobiol B: Biol, 35(3), 149-57; Merchat et al., 1996. J. Photochem. Photobiol. B: Biology, 32, 153-157, and references therein). Hematoporphyrin derivatives irradiated with a xenon lamp or a dye laser inactivated HSV, CMV, HIV and SIV in whole blood with minimal damage to red cells, to platelets or to complement factors (Matthews et al., 1992. Blood Cells, 18, 75-89), and a benzoporphyrin derivative eliminated vesicular stomatitis virus and feline leukemia virus from blood and blood products (North et al., 1992. Blood Cells, 18, 129-140), with the additional advantage of also killing virally-infected lymphocytes without apparent damage to red cells. Photofrin™ itself has been shown to inactivate HSV in buffer (Grandadam et a., 1992. Photodynamic Inactivation of Wild Type and Mutant Herpes Simplex Virus Type 1 (HSV-1) by Photofrin. in Photodynamic Therapy and Biomedical Lasers. Spinellis, P, Dal Fante, M, and Marchsini, R, (eds), Excerpta Medica, Amsterdam). Water-soluble hydroxy-substituted texaphyrin metal complexes and other porphyrin derivatives have been suggested to be effective against HIV and possibly HIV-infected cells (U.S. Pat. No. 5,432,171 to Sessler et al, 1995) (U.S. Pat. No. 5,192,788 to Dixon et al., 1993). The ability of porphyrins to inactivate HAV, HEV, or other non-enveloped human viruses has heretofore not been described.

When assessing a new procedure for virus inactivation in a blood component, the two main considerations are safety and efficacy. The first relates to preservation of structure and function of the blood component such that no adverse effects will result from its transfusion and its therapeutic activity will be maintained. The second consideration implies that virus inactivation in the blood component is complete and that the risk of infection is eliminated. This usually means >6 log₁₀ inactivation of the virus infectious dose. In addition, an understanding of the mechanism of action of the treatment is useful for subsequent evaluation as well as for optimization.

Effective methods to remove or inactivate viruses, bacteria, and parasites in blood would offer a greater measure of safety for the transfusion of blood components. These methods must be relatively inexpensive and easy to use. Ease of use would also be of value in cases of emergency and in underdeveloped countries where transfusion-transmitted disease prevalence is much greater. The development of effective disinfection methods would not only provide greater safety in the blood supply, but would also increase storage time for blood products.

Some studies have investigated the photosensitization of viruses in aqueous buffers containing added serum proteins, in factor VIII and factor IX protein solutions, in human plasma with and without platelets, and in whole blood or red blood cell concentrates. Although photochemical approaches for sterilization of the cellular blood components are still experimental, some methods, such as psoralen+UVA irradiation of platelet concentrates are underway in clinical trials in the U.S. (Ben-Hur and Horowitz, 1997. Photochem Photobiol 65(3), 427). In addition, the use of methylene blue photodynamic treatment of individual units of fresh plasma has been used by a number of Red Cross transfusion services in Germany and Switzerland since 1992 (Ben-Hur and Horowitz, 1995. Photochem Photobiol 62, 383-388; Ben-Hur and Horowitz, 1996. AIDS 10, 1183-1190; Mohr, 1997. Photochem Photobiol 65(3), 441-445).

Photoinactivation of microbial contaminants in blood, is described in, for example, U.S. Pat. No. 6,177,441 to Cooke et al; U.S. Pat. No. 6,194,139 to Wollowitzetal.; U.S. Pat. No. 6,010,890 to Ben-Hur et al.; U.S. Pat. No. 5,981,163 to Horowitz et al.; U.S. Pat. No. 5,985,331 to Gottleib et al.; U.S. Pat. No. 5,955,256 to Sowemimo-Coker et al.; U.S. Pat. No. 5,912,241 to Gottleib et al.; U.S. Pat. No. 5,869,701 to Park et al.; U.S. Pat. No. 5,932,468 to Debart; U.S. Pat. No. 5,712,086 to Horowitz et al.; U.S. Pat. No. 5,789,238 to Goodrich et al.; U.S. Pat. No. 5,780,287 to Kraus et al.; U.S. Pat. No. 5,776,966 to North; U.S. Pat. No. 5,736,563 to Richter; U.S. Pat. No. 5,837,519 to Savage et al.; U.S. Pat. No. 5,789,601 to Park et al.; U.S. Pat. No. 5,789,150 to Margolis-Nunno et al.; U.S. Pat. No. 5,597,722 to Chapman et al.; U.S. Pat. No. 5,516,629 to Park et al.; U.S. Pat. No. 5,545,516 to Wagner et al.; U.S. Pat. No. 5,587,490 to Goodrich, Jr., et al.; U.S. Pat. No. 5,527,704 to Wolf, Jr., et al.; U.S. Pat. No. 5,445,629 to Debrauwere et al.; U.S. Pat. No. 5,432,171 to Sessler et al.; U.S. Pat. No. 5,360,734 to Chapman et al.; U.S. Pat. No. 5,232,844 to Horowitz et al.; U.S. Pat. No. 5,192,788 to Dixon et al.; U.S. Pat. No. 5,120,649 to Horowitz et al.; U.S. Pat. No. 5,041,078 to Matthews et al.; U.S. Pat. No. 5,030,200 to Judy et al.; U.S. Pat. No. 4,878,891 to Judy et al.; and U.S. Pat. No. 4,748,120 to Wiesehahn.

Porphyrins and porphyrin technology is generally described in, for example, U.S. Pat. No. 6,005,087 to Cook et al.; U.S. Pat. No. 6,001,573 to Roelant et al.; U.S. Pat. No. 5,998,128 to Roelant et al.; U.S. Pat. No. 5,955,603 to Therien et al.; U.S. Pat. No. 5,952,311 to Kraus et al.; U.S. Pat. No. 5,891,689 to Takle et al.; U.S. Pat. No. 5,922,537 to Ewart et al.; U.S. Pat. No. 5,916,539 to Pilgrimm; U.S. Pat. No. 5,876,989 to Berg et al.; U.S. Pat. No. 5,786,219 to Zhang et al.; U.S. Pat. No. 5,714,328 to Magda et al.; U.S. Pat. No. 5,744,302 to Sessler et al.; U.S. Pat. No. 5,709,944 to Pease et a.; U.S. Pat. No. 5,595,726 to Magada et al.; and U.S. Pat. No. 5,660,731 to Piechocki et al. However, the use of second-generation porphyrins specifically for the inactivation of certain viruses and bacteria is not described.

### SUMMARY OF THE INVENTION

The present invention is provides methods for inactivation of microbial pathogens and viruses via photosensitization by porphyrins. Accordingly, in one aspect, the invention relates to a method of inactivating a microbial pathogen in a fluid medium by first contacting the fluid medium with a porphyrin, and then exposing the fluid medium to irradiation for an amount of time sufficient to inactivate the microbial pathogen. Preferred porphyrins are light-activated, meso-substituted, amphoteric porphyrins. Suitable microbial pathogens are viruses and bacteria, with small, non-enveloped viruses (e.g., hepatitis A virus (HAV), B19, poliovirus) being particularly preferred. Fluid media that may be treated by the methods of the present invention include drinking water, wastewater and blood products (e.g., plasma, red blood cells), with blood products being preferred.

In a preferred embodiment, the invention relates to a method for inactivating a virus in a blood product by contacting the blood product with a porphyrin, and then exposing the blood product to irradiation for an amount of time sufficient to inactivate the virus. A more preferred embodiment relates to a method of inactivating a non-enveloped virus in a blood product by contacting the blood product with a light-activated, meso-substituted, amphoteric porphyrin, and then exposing the blood product to ultraviolet light for an amount of time sufficient to inactivate the virus.

The foregoing and other aspects of the present invention are explained in detail in the specification set forth below.

### BRIEF DESCRIPTION OF THE DRAWINGS

FIG. 1 illustrates the structure of TMPyP4 (1) and TPPS4 (2).

FIG. 2 is a graph showing the inactivation of poliovirus in water containing 10⁻⁵ M TMPyP4 (cationic porphyrin). Closed squares denote PV1 in 10⁻⁵ M TMPyP4 and TPSS4 in water with no light. Diamonds denote PV1 in 10⁻⁵ M TMPyP4 and TPPS4 in water with light. Clear diamonds denote detection limit.

FIG. 3 is a graph showing the inactivation of coliphage MS2 in 0.2 M PBS or 0.14 M NaCl containing 10⁻³ or 10⁻⁵ M TMPyP4 (cationic) or TPPS4 (anionic) porphyrin. Diamonds with solid line denote MS2 in 10⁻³ M TMPyP4 in PBS with light. Diamonds with dashed line denote MS2 in 10⁻⁵M TPPS4 in PBS with no light. Squares denote MS2 in 10⁻³ M TMPyP4 in NaCl with light. Triangles denote MS2 in 10⁻⁵ M TMPyP4 in PBS with light. “X's” denote MS2 in 10⁻⁵M TPPS4 in PBS with light. Asterisks denote MS2 in 10⁻⁵M TPPS4 in NaCl with light. Diamonds denote MS2 in 10⁻⁵ M TPPS4 in PBS with no light. Clear symbols denote detection limit.

FIG. 4 is a graph showing the inactivation of coliphage MS2 in human plasma containing 10⁻⁵ M TPPS4 (anionic) porphyrin. Diamonds denote MS2 in 10⁻⁵ M TPPS4 in plasma with no light. Squares denote MS2 in 10⁻⁵ M TPPS4 in plasma with light. Clear symbols denote detection limit.

FIG. 5 is a graph showing the inactivation of HAV in water containing 10⁻⁵ M TMPyP4 (cationic) or TPPS4 (anionic) porphyrin. Plus symbols denotes HAV in 10⁻⁵ M TPPS4 in water with no light. Asterisks denote HAV in 10⁻⁵ M TMPyP4 in water with no light. Diamonds denote HAV in 10⁻⁵ M TMPyP4 in water with light. Squares denote HAV in 10⁻⁵ M TPPS4 in water with no light. Clear symbols denote detection limit.

FIG. 6 is a graph showing the inactivation of HAV in human plasma containing 10⁻⁴ or 10⁻⁵ M TPPS4 (anionic) porphyrin. Diamonds denote HAV in 10⁻⁵ TPPS4 in plasma with light and squared denote HAV in 10⁻⁴ TPPS4 in plasma with light.

FIG. 7 is a graph showing the inactivation of Klebsiella oxytoca in human plasma containing 10⁻⁵ M TMPyP4 (cationic) porphyrin. Diamonds denote K. oxytoca in 10⁻⁵ M TMPyP4 in plasma with light and squares denote K. oxytoca in 10⁻⁵ M TMPyP4 in plasma with no light. Clear symbols denote detection limit.

FIG. 8 is a graph showing the inactivation of E. coli B on porphyrin-containing cellulose acetate.

FIG. 9 is a graph showing the inactivation of E. coli B in 0.2 M PBS containing 10⁻³ or 10⁻⁵ M TMPyP4 (cationic) or TPPS4 (anionic) porphyrin. Diamonds denote E. coli in 10⁻³ M TMPyP4 in PBS with light. Squares denote E. coli in 10⁻⁵M TMPyP4 in PBS with light. Triangles denote E. coli in 10⁻⁵ M TPPS4 in PBS with light. Plus symbols denote E. coli in PBS only with light. Asterisks denote E. coli in 10⁻⁵ M TPPS4 in PBS with no light. Clear symbols denote detection limit.

### DETAILED DESCRIPTION OF PREFERRED EMBODIMENTS

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

The present invention generally relates to methods of inactivating microbial pathogen in fluid media. In a broad sense, the methods comprise contacting a fluid medium with a light-activated porphyrin; and then exposing the fluid medium to irradiation for an amount of time sufficient to inactivate the microbial pathogen.

“Inactivation” of a microbial pathogen may mean, interchangeably, destroying the population of the pathogen in the medium, or may mean significantly decreasing the presence or concentration of the pathogen in the medium. Inactivation also refers to destroying or decreasing the activity (e.g., infectivity, virulence, transmissibility, pathogenicity) of the microbial pathogen.

One measure of inactivation is a decrease in detectable activity of the pathogen as measured in a decrease of the virus infectious dose in log₁₀ units. For example, inactivation may mean a greater than (>) 3 log₁₀ inactivation of the virus infective dose, preferably >4 log₁₀, more preferably >5 log₁₀, and even more preferably >6 log₁₀. Alternatively, inactivation may be measured in percentages; that is, inactivation may mean a decrease of greater than 90% of activity of the pathogen, preferably a decrease of greater than 95% of activity of the pathogen; more preferably a decrease of greater than 98% of activity of the pathogen; and even more preferably a decrease of greater than 99% of activity of the pathogen.

Fluid media that may be treated by the present invention includes but is not limited to drinking water, wastewater, buffered aqueous solutions (e.g., comprising proteins or other biological compounds), and bodily fluids (blood, blood products, urine, serum, cerebrospinal fluid, saliva). In a preferred embodiment, the fluid medium is a bodily fluid; in a more preferred embodiment, the fluid medium is a blood product (including blood). In one preferred embodiment, the fluid medium comprises a cell, and more preferably, a mammalian cell.

The term “blood product” includes but is not limited to liquid blood products such as blood, whole blood, blood fractions, plasma (both comprising platelets or devoid of platelets), plasma derivatives, whole blood concentrates, red blood cell concentrates, white blood cell concentrates, serum, clotting factors, platelet concentrates, cryoprecipitated antihemophilic factor (AHF), blood protein solutions (e.g., plasma proteins in solution, coagulation/clotting factors in solution), and others. The blood products may be fresh or thawed. Preferably the blood products comprise blood plasma (e.g., blood plasma, whole blood). Preferred blood products are red blood concentrates and plasma. Other preferred fluid media includes aqueous buffers comprising at least one serum protein (e.g., factor VIII and factor IX) or at least one plasma protein (e.g., antibodies, immunoglobulins, coagulation factors).

When the present invention is used in the treatment of blood products, the treatment of human blood products is preferred. However, it will be appreciated that the present invention may be carried out on blood products collected from other animals, particularly products from mammalian species such as dogs, cats, rabbits, horses, goats, and cattle, for veterinary purposes, for the development and manufacture of biological products, and for drug screening and development purposes.

The fluid medium may be known to contain microbial pathogens or viruses, or alternatively may be one suspected of containing such microbial pathogens or viruses, or a product in which contamination is unknown or not suspected by where the photoinactivation procedure described herein is performed as a precautionary step.

Microbial pathogens that may be inactivated by the methods of the present invention include viruses and bacteria. Preferred pathogens are viruses, with small, non-enveloped viruses being more preferred. “Small” viruses are those viruses that have an approximate virion diameter of less than about 30 nm, preferably less than about 27 nm, more preferably less than about 25 nm, about 24 nm, or about 23 nm. In general, a small virus is intended to mean a virus whose virion diameter is from about 22 nm to about 27 nm. The term “non-enveloped virus” has the meaning generally applied in the art, that is, a virus whose virions lack the lipid bilayer and associated glycoprotein envelope associated with many enveloped virus types.

Particular viruses that may be inactivated by the present invention include but are not limited to viruses in the family Parvoviridae (including human, animal and insect-infecting parvoviruses, such as parvovirus B19 and adeno-associated virus), hepatitis A virus (HAV), hepatitis E virus (HEV), polioviruses, and coliphages such as MS2. Viruses with RNA genomes (i.e., comprise RNA, either single-stranded or double-stranded) and DNA genomes (i.e., comprise DNA, either single-stranded or double-stranded) may be inactivated by the methods of the invention.

Bacteria that may be inactivated by the present invention include but are in no way limited to Staphylococcus epidermidis, Bacillus spp., bacteria of the Escherichia genus and bacteria of the Klebsiella genus. In particular embodiments, the invention is useful in inactivating Escherichia coli. and Klebsiella bacteria, including Klebsiella oxytoca.

Porphyrins are known in the art. In preferred embodiments, the porphyrin is a light-activated, meso-subsituted porphyrin. In even more preferred embodiments, the porphyrin is an amphoteric (e.g., anionic or cationic) porphyrin. Exemplary porphyrins include but are not limited to(i) tetrakis (N-methyl-4-pyridiniumyl) porphine tetratosylate), abbreviated H₂TMPyP4; (ii) meso-tetra-(4-sulfonatophenyl)-porphine dihydrochloride (C₄₄H₃₂N₄O₁₂S₄Cl₂; MW 1007.69) abbreviated H₂TPPS4; (iii) tetrakis (4-n-butylpyridiniumyl) porphyrin, abbreviated TPyPH₂(N-Bu), MW 1166); and (iv) tetra-N-octyl tetrakis pyridinium porphyrin, abbreviated TPyPH₂(N-Oc). H₂TPPS4 is a sulfonated, negatively-charged compound, while H₂TMPyP4 has an overall positive charge. Other useful porphyrins include protoporphyrin IX. Certain porphyrins useful in the practice of the present invention are commercially available from, for example, Porphyrin Products (Logan, Utah).

In preferred embodiments, the porphyrin concentration in the fluid medium after the contacting step is from about 10⁻²M to about 10⁻⁵ M. In other embodiments, the porphyrin concentration in the fluid medium after the contacting step is from about 10³ M to about 10⁻⁵ M.

Porphyrins may advantageously be immobilized, incorporated into or absorbed onto solid-phase matrices such as polymers, plastics (formed into, e.g., tubing or bags), microbeads, resins, membranes, or other solid-phase formats. In certain embodiments, the solid phase matrix is a membrane (e.g., a cellulose acetate membrane or “CAM”).

After contacting the fluid medium with a porphyrin, the fluid medium is irradiated (i.e., exposed to light) for a length of time sufficient to inactivate the pathogen. Preferably, the irradiation is ultraviolet (UV) light (e.g., from about 4 nm to about 400 nm), and more preferably is long-wavelength UV (LWUV) light (e.g., from about 300 nm to about 400 nm). In a preferred embodiment, the wavelength of the LVUV light is from about 340 nm to about 380 nm, and more preferably is about 365 nm. In preferred embodiment, exposing the fluid medium comprising the porphyrin causes the generation of singlet oxygen.

The examples, which follow, are set forth to illustrate the present invention, and are not to be construed as limiting thereof.

### EXAMPLE 1

### Materials and Methods

Materials.

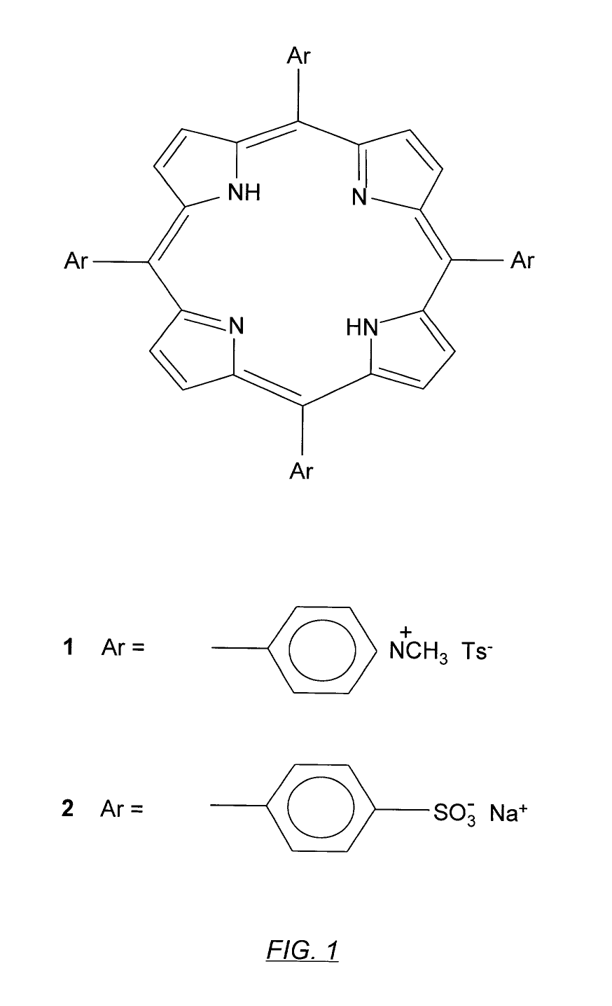

A portable UV lamp (Spectroline ENF-260C; Spectronics Corp., NY) was set to emit long-wavelength (365 nm) UV irradiation. TMPyP4 (1) or TPPS4 (2) (FIG. 1) were dissolved in 0.2 M phosphate-buffered saline pH 7.2 (PBS), 0.14 M saline solution pH 7.0 (NaCl), distilled, deionized, sterile water pH 7.0 (water), or in human plasma, adjusted to final concentrations of 0.001, 0.0001, or 0.00001 M porphyrin in medium. Porphyrins tested were (i) tetrakis (N-methyl-4-pyridiniumyl) porphine tetratosylate), abbreviated H2TMPyP4; (ii) meso-tetra-(4-sulfonatophenyl)-porphine dihydrochloride (C₄₄H₃₂N₄O₁₂S₄Cl₂; MW 1007.69) abbreviated H₂TPPS4; (iii) tetrakis (4-n-butylpyridiniumyl) porphyrin, abbreviated TPyPH₂(N-Bu), MW 1166); and (iv) tetra-N-octyl tetrakis pyridinium porphyrin, abbreviated TPyPH₂(N-Oc). H₂TPPS₄ is a sulfonated, negatively-charged compound, while H₂TMPyP₄ has an overall positive charge.

Human Plasma.

Fresh, frozen human plasma (Type O, Rh positive; from the University of North Carolina Hospitals Transfusion Service) was stored at −20° C. Plasma bags were thawed in a 37° C. water bath. Thawed plasma was aliquoted in 40-50 mL portions then used immediately or re-frozen.

Experimental Procedures.

The cytopathic variant of HAV, strain HM175, was grown and assayed by the plaque technique in newly confluent layers of FRhK-4 (fetal rhesus kidney-derived) cells as previously described (Cromeans, et al., 1987. J Med Virol 22, 45-56). Poliovirus-1 was propagated in BGMK (African Green Monkey Kidney-Derived) cells and assayed for infectivity by the plaque technique as previously described (Sobsey et al., 1978. Appl Envr Microbiol 36, 121), while assay and growth of coliphage MS2 (ATCC 15597-B1) was based on the top agar overlay plaque method (Adams, 1959. Bacteriophages. Wiley-Interscience, New York). Virus-containing stocks were desalted by centrifugal ultrafiltration in Centricon-100 ultrafilters (Amicon, Inc., Beverly, Mass.) that had been sanitized with 70% ethanol and pretreated with 0.1% Tween-80 in PBS. Aliquots of stock virus were ultrafiltered at 1000× g and the dead stop volume was replenished with sterile water for a total of three times. To ensure monodispersion of virions, the desalted portions were filtered successively through 0.01% Tween-80 treated 0.2 and 0.08 μm pore size polycarbonate (Nucleopore) filters. Filtered and purified stocks were stored at 4° C.

Cultures of E. coli B (ATCC 11303) were grown the day before an experiment to log phase growth in 50 milliliters of tryptic soy broth (TSB) followed by centrifugation at 3000 rpm for 10 minutes and at 4° C. The supernatant was drawn off and discarded and pellets were resuspended in 10 mL of 0.2 M phosphate buffered saline (PBS; pH 7.2). The centrifugation-resuspension process was repeated for a total of three times. Preparations were kept at 4° C. until ready for use. Bacterial cultures were assayed on tryptic soy agar (TSA) by spot or spread plating duplicate 0.04 to 0.1 ml volumes of the serially diluted preparation onto the agar plates, and was enumerated via colony counts after incubation overnight at 44.5° C. Results are expressed as cfu/ml. On the day of an experiment, the stock preparation was serially diluted in PBS to a target concentration of about 104 or 108 E. coli cells per milliliter for use on membranes and in solution, respectively. Duplicate 0.1 ml volumes of the experimental samples were spread plated on TSA and incubated overnight at 44.5° C.

Toxicity assays consisted of adding porphyrins to dishes containing Chinese Hamster Ovary (CHO) cells. CHO cells were seeded into a six-well tissue culture plates and maintained in Eagle's Minimum Essential Medium (1×) containing 10% heat-inactivated fetal calf serum, 1% L-glutamine, 1% non-essential amino acids, and 1% gentamycin/kanamycin. Concentrated porphyrin solutions were added to the existing maintenance media to the desired final concentration, incubated for 5 days at 37° C. and 5% CO2, and the cells were stained using crystal violet dye. Colony-forming units (cfu) of cells were enumerated and compared to the numbers of cells not exposed to porphyrins.

Photosensitization experiments were conducted at room temperature under a laminar-flow hood. A UV lamp (Spectroline ENF260C; Spectronics Corp., NY) was the light source in these experiments and was set for long-wavelength (365 nm) UV irradiation. Porphyrin-containing solutions were magnetically mixed in petri dishes containing stir bars. The UV lamp unit was placed 3.5 cm above the dishes, and the test viruses were added to the porphyrin solutions and petri dishes to a final concentration of 104 to 106 plaque-forming units (pfu) per milliliter. Control samples consisted of porphyrin and microbes not exposed to light, and of microbes exposed to light without porphyrins. The UV light was turned on to begin an experiment. At 1, 10, 30, 60, or 90 minutes, 0.5 mL aliquots were withdrawn from the petri dishes and then stored on ice in the dark. At the beginning and end of each experiments, 0.5 mL aliquots of microbes in the porphyrin solution not exposed to light or microbes in test solvents were withdrawn and kept on ice. Timed samples were serially diluted in appropriate diluents (PBS for bacteria and coliphage MS2; cell culture diluent for HAV and PV-1) or in test solvents, from 1:10 to 1:10000 and assayed. Results are expressed as the percent reduction in log10 plaque-forming units (pfu) of infectious virus at time (t) when compared to numbers of viruses in porphyrin solutions not exposed to treatment with UV light.

### EXAMPLE 2

### Inactivation of Poliovirus

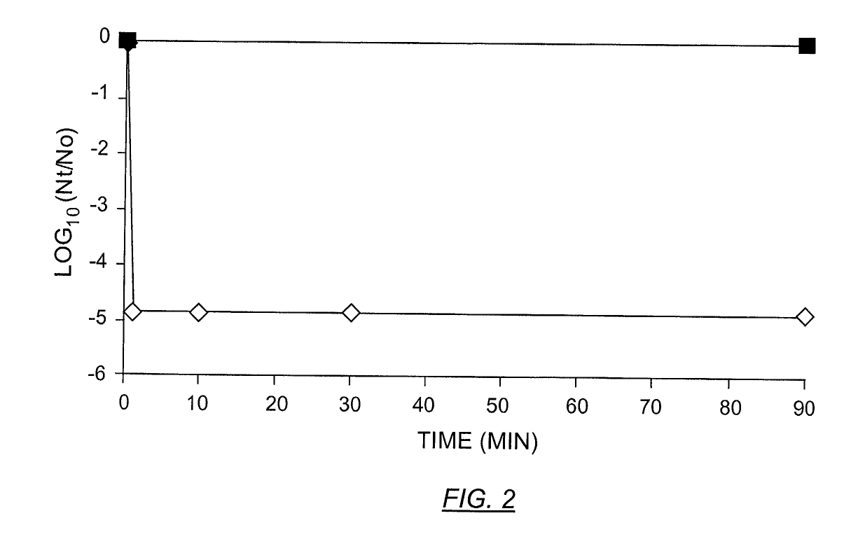

Irradiation of porphyrin-containing solutions with long-wavelength UV light effectively reduces numbers of infectious microorganisms. This is graphically demonstrated by FIG. 2, which shows poliovirus (PV1) in water is rapidly inactivated below the limits of detectability, >4.1 log₁₀ (>99.99%), in 1 minute by 10⁻⁵ M TMPyP4 (1) in the presence of light, but is unaffected in the absence of light. Similar results were obtained with 10⁻⁵M TPPS4 (2), results not shown. Inactivation levels denoted as greater than (>) the reported value (e.g., >99.97%) indicates that the level of inactivation may actually have been greater than observed, since the lower limit of detection for the assay was reached.

### EXAMPLE 3

### Inactivation of the Non-enveloped Bacteriophage MS2

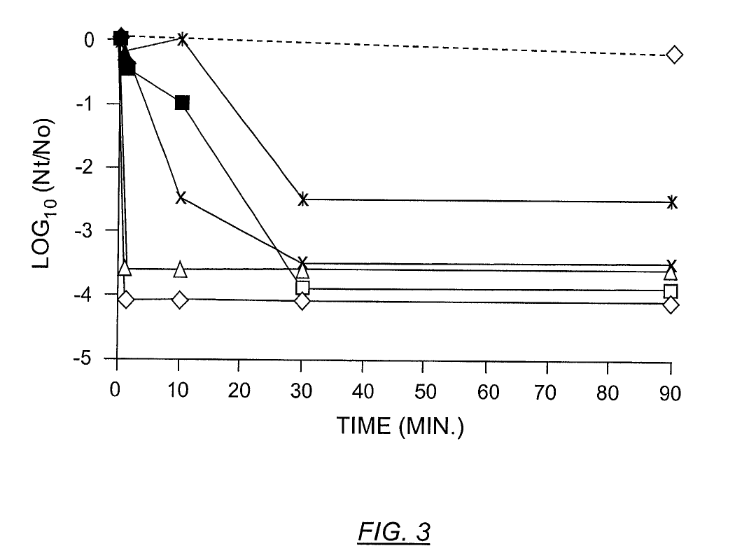

The non-enveloped bacteriophage MS2 was also inactivated by both TMPyP4 (1) and TPPS4 (2), and unaffected in the absence of light, as shown in FIG. 3. This experiment also included a comparison of the effects of different buffers. MS2 treated with 10⁻³ M TMPyP4 in NaCl was inactivated to 4-5 log₁₀ (99.99-99.999%) in 30 minutes after exposure to UV light. Specifically, log₁₀ reductions of MS2 in TMPyP4 in NaCl were 0.3 (50%) in 1 minute, 0.9 (87%) in 10 minutes, and >3.8 (>99.98%) in 60 minutes. Microbes treated with TPPS4 (2) in PBS which were not exposed to UV light showed no loss of infectivity; approximately 100% of the initial infectious test microbes remained at the end of each experimental period. Specifically, log₁₀ reductions in TPPS4 in PBS were 0.2 (37%), 2.5 (99.7%), and >3.5 (>99.97%) in 1, 10, and 60 minutes, respectively. The cationic porphyrin TMPyP4 (1) appeared on the whole more potent than the anionic porphyrin TPPS4 (2).

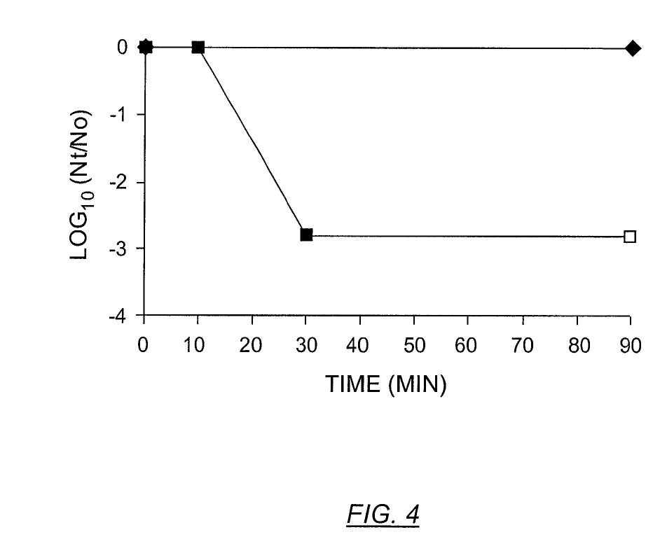

FIG. 4 demonstrates that TPPS4 (2) was about as effective in inactivation of coliphage MS2 in plasma (about 3 logs at 30 min and 10⁻⁵M) as it was in PBS or in saline (FIG. 3). At a concentration of 10⁻⁵ M TPPS4/plasma irradiated with LWUV, coliphage MS2 was unaffected after 10 minutes of contact, but was then inactivated by >2.8 log₁₀ (>99.8%) in 30 minutes. Reductions of coliphage MS2 in 10⁻⁵ M TMPyP4/plasma was >99.5% (>2.3 log₁₀) in 1 minute. However, MS2 added to the TMPyP4/plasma mixture and not exposed to LWUV was not detectable. Similarly, inactivation of 4 to 5log₁₀ (99.99-99.999%) for MS2 and E. coli B suspended in 10⁻³ M TMPyP4/PBS was observed in 1 minute after exposure to UV light. However, these microbes incubated with 10⁻³ M TMPyP4/PBS in the dark were not detected via infectivity assay. It is uncertain whether the levels of inactivation observed were due to photosensitization or some as yet unrecognized effect involving the porphyrin and its solvent.

### EXAMPLE 4

### Inactivation of the Non-enveloped Virus HAV

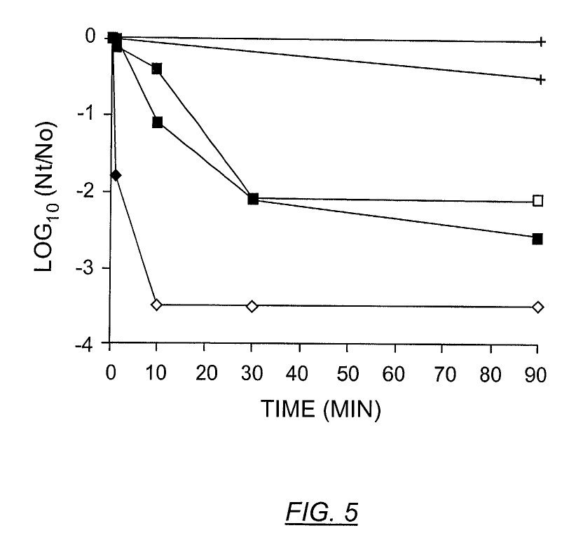

The activity of the two porphyrins against the non-enveloped virus HAV was compared. The results are shown graphically in FIG. 5. FIG. 5 indicates that HAV is more completely inactivated in water by the cationic porphyrin TMPyP4 (1) (10⁻⁵M, greater than 3 logs in 30 min) than by the anionic porphyrin TPPS4 (2) (10⁻⁵M), which achieved only 2 logs reduction in the same time.

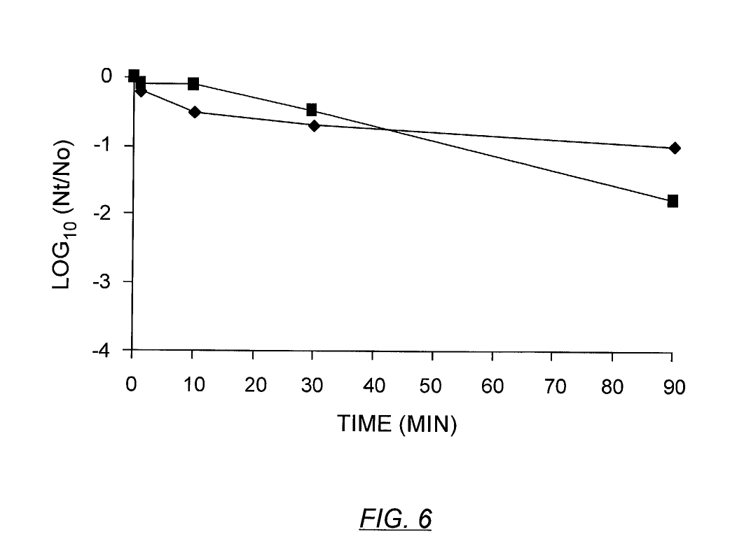

When HAV was exposed to TPPS4 (2) and light in plasma (FIG. 6), only about 1 log of inactivation was seen at 10⁻⁵M, and an increase in concentration (10⁻⁴M) and time (90 min) were required to reach 2 logs reduction, in contrast to the results with MS2, which was inactivated equally well by TPPS4 (2) in plasma (FIG. 4) or in buffer (FIG. 3). At a concentration of 10⁻⁵ M TPPS4 in plasma, reductions of HAV were 0.2, 0.5, 0.7, and 1 log₁₀ (90%) in 1, 10, 30, and 90 minutes, respectively. At a higher concentration of 10⁻⁴ M TPPS4 in plasma, reductions were similar to the 10⁻⁵ M concentration at 1, 10, and 30 minutes, with 0.1, 0.1, and 0.5 log₁₀, reductions, respectively, but was more extensive overall (>1.8 log₁₀ or >98% at 90 minutes). The level of inactivation observed for HAV may have actually been higher than observed, as the detection limits were reached at 90 minutes at the higher (10⁻⁴ M) concentration of TPPS4 in plasma. In contrast to the reduction of HAV by TPPS4 in plasma, inactivation of HAV by TPyPH2(N-Bu) in plasma was more extensive. At a concentration of 10⁵⁻M TPyPH2(N-Bu) in plasma, reductions of HAV were 0.1, 1.5, 1.6, 2.7, and >3.3 log₁₀ in 1, 10, 30, and 90 minutes, respectively.

### EXAMPLE 5

### Inactivation of Bacteria

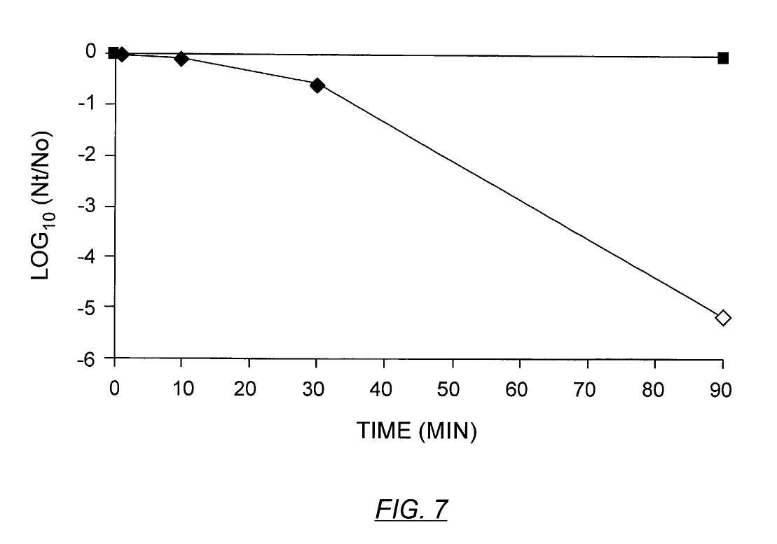

FIG. 7 shows that TMPyP4 was also able to achieve over 5 log₁₀ inactivation of the gram-negative enterobacterium Klebsiella oxytoca in plasma.

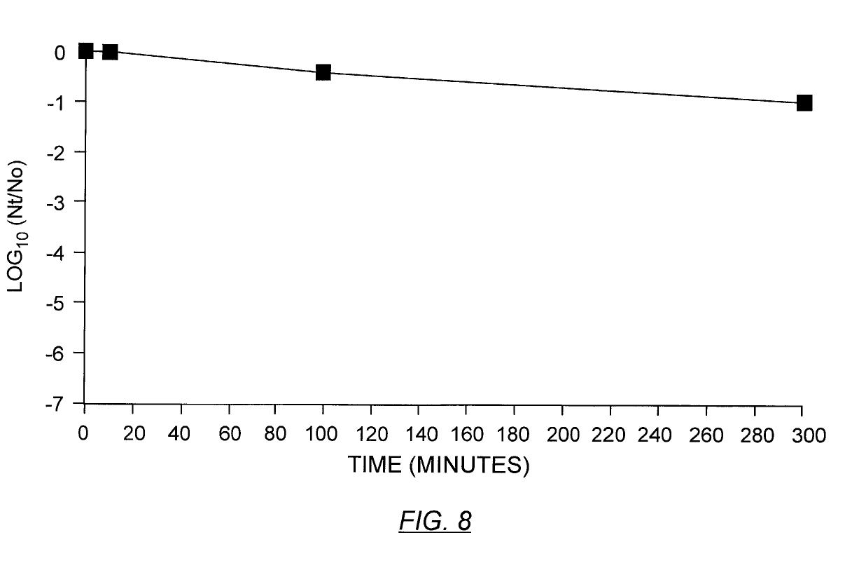

When cellulose acetate membranes containing protoporphyrin IX (disodium salt) were exposed to UV light, E. coli B was inactivated by 0.3 log₁₀ cfu/ml in 100 minutes and by about 1.0 log₁₀ (90%) in 300 minutes (FIG. 8). No change in concentration of E. coli was observed on membranes not exposed to UV light. Additional experiments suggested that decreasing the distance of the UV light from the membrane, delivering air into the microbe-containing inoculum on the surface of the membrane, and irradiation or contact times of 100 minutes or greater were necessary for demonstrable inactivation of E. coli.

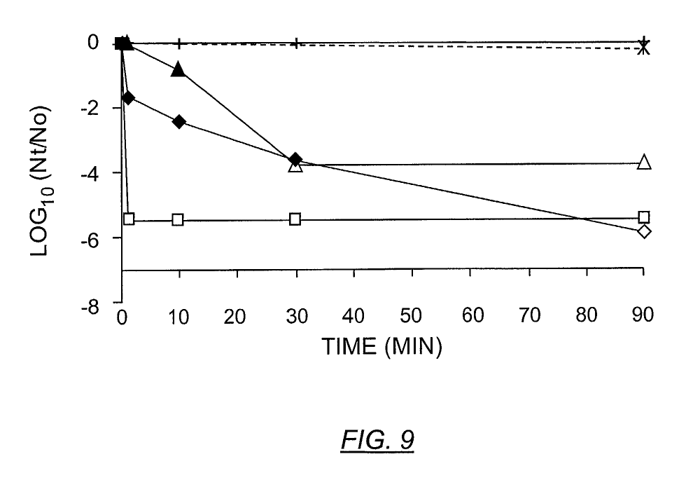

Biocidal activity was next evaluated in solution. Infectivity was decreased below detection limits after one minute of treatment with 10⁻⁵ M TMPyP4 (1) in PBS, with or without light (FIG. 9). With 10⁻³ M TMPyP4 (1) in NaCl, log₁₀ (percentage) reductions were 1.7 (98%), 2.4 (99.6%), 3.7 (99.98%), and >5.9 log₁₀ (>99.999%) after 1, 10, 30, and 90 minutes of exposure to long-wavelength UV light (365 nm), respectively. Treatment of Escherichia coli B with 10⁻⁵ M TPPS4 (2) in PBS under 365 nm light produced log₁₀ reductions of 0.6 (75%) and 1.6 (97%) after 1 and 10 minutes, and >5.5 log₁₀ (>99.999%) in 30 minutes. Treatment with either TPPS4 (2) in the absence of long-wavelength UV light, or light in the absence of porphyrin, did not result in any loss of infectivity.

Initial studies with the commercially-available positively-charged (cationic) porphyrin mesotetrakis (N-methyl-4-pyridiniumyl) porphyrin tetratosylate (1) showed that over 3.7 log inactivation of Escherichia coli B could be achieved in 30 minutes when the medium was aerated by sparging with air. No inactivation was observed when the medium was not aerated. Light alone, or porphyrin alone, were ineffective. These results support the hypothesis that porphyrins can serve as photo-activated anti-microbial agents, and act by production of singlet oxygen.

### EXAMPLE 6

### Toxicity of Porphyrins on Mammalian Cells

To test the toxicity of porphyrins on mammalian cells, TMPyP₄ and TPPS₄ dissolved in PBS were added to cell culture media to final concentrations of 10⁻³, 10⁻⁴, or 10⁻⁵ M. Controls consisted of culture media only. After incubation for six days followed by crystal violet staining, the average number of cfu/well for the controls was 271 (range, 256-308). In contrast, a concentration of 10⁻³ M of either TPPS4 or TMPyP4 were completely toxic to CHO cells (i.e., <1 cfu/well). However, counts of 320 and 310 cfu/well were recorded for concentrations of 10⁻⁴ or 10⁻⁵ M of TPPS4, respectively. TMPyP4 at a molar concentration of 10⁻⁵ yielded counts of 322 cfu/well, but at a concentration of 10⁻⁴M, no cells could be detected. In another experiment, concentrated TPyPH₂(N-Bu), TMPyP4, or TPPS4 were added to cell culture media for final concentrations of 10⁻⁴ or 10⁻⁵ M. Control wells consisted of culture media only. The number (mean) of colony forming units/well for controls was 193 cfu/well (range, 190-196). At concentrations of 10⁻⁴ M TPyPH₂(N-Bu) or TMPyP₄, there were no colony forming units detected. In contrast, TPPS4 added to a concentration of 10⁻⁴ M, and TPyPH2(N-Bu) or TMPyP4 added to a concentration of 10⁻⁵ M, had slightly lower counts when compared to controls. The average cfu/well for 10⁻⁴ M TPPS4 was 189 cfu/well, while the average counts for 10⁻⁵ M TPyPH2(N-Bu) or TMPyP4 were 186 cfu/well and 183 cfu/well, respectively.

The foregoing examples are illustrative of the present invention, and are not to be construed as limiting thereof. The invention is described by the following claims, with equivalents of the claims to be included therein.

What is claimed is:

1. A method of inactivating a microbial pathogen in a fluid medium, comprising:
contacting the fluid medium with a porphyrin; and then
exposing the fluid medium to irradiation for an amount of time sufficient to inactivate the microbial pathogen.

2. The method according to claim 1, wherein the fluid medium is a bodily fluid.

3. The method according to claim 1, wherein the bodily fluid comprises a cell.

4. The method according to claim 1, wherein the bodily fluid comprises a mammalian cell.

5. The method according to claim 1, wherein the fluid medium is a blood product.

6. The method according to claim 5, wherein the blood product is selected from the group consisting of plasma, serum, whole blood concentrates, red blood cell concentrates, white blood concentrates, clotting factor, and platelet concentrates.

7. The method according to claim 5, wherein the blood product comprises an aqueous buffer comprising at least one serum protein.

8. The method of claim 7, wherein the protein is selected from the group consisting of factor VIII and factor IX.

9. The method according to claim 5, wherein the blood product comprises a protein solution.

10. The method according to claim 9, wherein the protein solution comprises a serum protein selected from the group consisting of factor VIII and factor IX.

11. The method according to claim 5, wherein the blood product is plasma.

12. The method according to claim 5, wherein the blood product is human plasma.

13. The method according to claim 5, wherein the blood product is plasma comprising platelets.

14. The method according to claim 5, wherein the blood product is plasma devoid of platelets.

15. The method according to claim 5, wherein the blood product comprises red blood cells.

16. The method according to claim 5, wherein the blood product comprises white blood cells.

17. The method according to claim 1, wherein the microbial pathogen is a virus.

18. The method according to claim 17, wherein the virus is a non-enveloped virus.

19. The method according to claim 17, wherein the virus comprises RNA.

20. The method according to claim 17, wherein the virus comprises DNA.

21. The method according to claim 17, wherein the virus is selected from the group consisting of hepatitis A virus (HAV), parvovirus B19, poliovirus, and coliphage MS2.

22. The method according to claim 17, wherein the virus is HAV.

23. The method according to claim 1, wherein the microbial pathogen is a bacterium.

24. The method according to claim 23, wherein the bacterium is selected from the group consisting of bacteria of the Escherichia genus and bacteria of the Klebsiella genus.

25. The method according to claim 23, wherein the bacterium is Escherichia coli.

26. The method according to claim 23, wherein the bacterium is Klebsiella oxytoca.

27. The method according to claim 1, wherein the irradiation is ultraviolet (UV) light.

28. The method according to claim 1, wherein the irradiation is long wavelength UV (LWUV) light.

29. The method according to claim 1, wherein exposing the fluid to irradiation creates singlet oxygen.

30. The method according to claim 1, wherein the porphyrin is a meso-subsituted porphyrin.

31. The method according to claim 1, wherein the porphyrin is an amphoteric porphyrin.

32. The method according to claim 1, wherein the porphyrin is an anionic porphyrin.

33. The method according to claim 1, wherein the porphyrin is a cationic porphyrin.

34. The method according to claim 1, wherein the porphyrin is selected from the group consisting of tetrakis (N-methyl-4-pyridiniumyl) porphine tetratosylate) (H₂TMPyP4); meso-tetra-(4-sulfonatophenyl)-porphine dihydrochloride (H₂TPPS4); tetrakis (4-n-butylpyridiniumyl) porphyrin (TPyPH₂(N-Bu)); and tetra-N-octyl tetrakis pyridinium porphyrin (TPyPH₂(N-Oc)).

35. The method according to claim 1, wherein the porphyrin is protoporphyrin IX.

36. The method according to claim 1, wherein the porphyrin concentration in the fluid medium after the contacting step is from about 10⁻²M to about 10⁻⁵ M.

37. The method according to claim 1, wherein the porphyrin concentration in the fluid medium after the contacting step is from about 10⁻³ M to about 10⁻⁵ M

38. The method according to claim 1, wherein the contacting step comprises contacting the blood product with a solid matrix onto which a porphyrin has been absorbed.

39. The method according to claim 1, wherein the fluid medium is selected from the group consisting of drinking water and wastewater.

40. A method of inactivating a virus in a blood product, comprising:
contacting the blood product with a porphyrin; and then
exposing the blood product to irradiation for an amount of time sufficient to inactivate the virus.

41. The method according to claim 40, wherein the blood product is selected from the group consisting of plasma, serum, whole blood concentrates, red blood cell concentrates, white blood concentrates, clotting factor and platelet concentrates.

42. The method according to claim 40, wherein the blood product comprises an aqueous buffer comprising at least one serum protein.

43. The method of claim 42, wherein the protein is selected from the group consisting of factor VIII and factor IX.

44. The method according to claim 40, wherein the blood product comprises a protein solution.

45. The method according to claim 44, wherein the protein solution comprises a serum protein selected from the group consisting of factor VIII and factor IX.

46. The method according to claim 40, wherein the blood product is plasma.

47. The method according to claim 40, wherein the blood product is human plasma.

48. The method according to claim 40, wherein the blood product is plasma comprising platelets.

49. The method according to claim 40, wherein the blood product is plasma devoid of platelets.

50. The method according to claim 40, wherein the blood product comprises red blood cells.

51. The method according to claim 40, wherein the blood product comprises white blood cells.

52. The method according to claim 40, wherein the virus is a non-enveloped virus.

53. The method according to claim 40, wherein the virus has a size that is smaller than about 30 nm.

54. The method according to claim 40, wherein the virus comprises RNA.

55. The method according to claim 40, wherein the virus comprises DNA.

56. The method according to claim 40, wherein the virus is selected from the group consisting of hepatitis A virus (HAV), parvovirus B19, poliovirus, and coliphage MS2.

57. The method according to claim 40, wherein the virus is HAV.

58. The method according to claim 40, wherein the irradiation is ultraviolet (UV) light.

59. The method according to claim 40, wherein the irradiation is long wavelength UV (LWUV) light.

60. The method according to claim 40, wherein exposing the fluid to irradiation creates singlet oxygen.

61. The method according to claim 40, wherein the porphyrin is a meso-subsituted porphyrin.

62. The method according to claim 40, wherein the porphyrin is an amphoteric porphyrin.

63. The method according to claim 40, wherein the porphyrin is an anionic porphyrin.

64. The method according to claim 40, wherein the porphyrin is a cationic porphyrin.

65. The method according to claim 40, wherein the porphyrin is selected from the group consisting of tetrakis (N-methyl-4-pyridiniumyl) porphine tetratosylate) (H₂TMPyP4); meso-tetra-(4-sulfonatophenyl)-porphine dihydrochloride (H₂TPPS4); tetrakis (4-n-butylpyridiniumyl) porphyrin (TPyPH₂(N-Bu)); and tetra-N-octyl tetrakis pyridinium porphyrin (TPyPH₂(N-Oc)).

66. The method according to claim 40, wherein the porphyrin is protoporphyrin IX.

67. The method according to claim 40, wherein the porphyrin concentration in the fluid medium after the contacting step is from about 10⁻²M to about 10⁻⁵ M.

68. The method according to claim 40, wherein the porphyrin concentration in the fluid medium after the contacting step is from about 10⁻³ M to about 10⁻⁵ M

69. The method according to claim 40, wherein the contacting step comprises contacting the blood product with a solid matrix onto which a porphyrin has been absorbed.

70. A method of inactivating a non-enveloped virus in a blood product, comprising:
contacting the blood product with a light-activated, meso-substituted, amphoteric porphyrin; and then
exposing the blood product to ultraviolet light for an amount of time sufficient to inactivate the virus.

71. The method according to claim 70, wherein the blood product is selected from the group consisting of plasma, serum, whole blood concentrates, red blood cell concentrates, white blood concentrates, clotting factor and platelet concentrates.

72. The method according to claim 70, wherein the blood product comprises an aqueous buffer comprising at least one serum protein.

73. The method of claim 72, wherein the protein is selected from the group consisting of factor VIII and factor IX.

74. The method according to claim 70, wherein the blood product comprises a protein solution.

75. The method according to claim 74, wherein the protein solution comprises a serum protein selected from the group consisting of factor VIII and factor IX.

76. The method according to claim 70, wherein the blood product is plasma.

77. The method according to claim 70, wherein the blood product is human plasma.

78. The method according to claim 70, wherein the blood product is plasma comprising platelets.

79. The method according to claim 70, wherein the blood product is plasma devoid of platelets.

80. The method according to claim 70, wherein the blood product comprises red blood cells.

81. The method according to claim 70, wherein the blood product comprises white blood cells.

82. The method according to claim 70, wherein the virus has a size that is smaller than about 30 nm.

83. The method according to claim 70, wherein the virus comprises RNA.

84. The method according to claim 70, wherein the virus comprises DNA.

85. The method according to claim 70, wherein the virus is selected from the group consisting of hepatitis A virus (HAV), parvovirus B19, poliovirus, and coliphage MS2.

86. The method according to claim 70, wherein the virus is HAV.

87. The method according to claim 70, wherein the UV light is long wavelength UV (LWUV) light.

88. The method according to claim 70, wherein exposing the fluid to UV light creates singlet oxygen.

89. The method according to claim 70, wherein the porphyrin is an anionic porphyrin.

90. The method according to claim 70, wherein the porphyrin is a cationic porphyrin.

91. The method according to claim 70, wherein the porphyrin is selected from the group consisting of tetrakis (N-methyl-4-pyridiniumyl) porphine tetratosylate) (H₂TMPyP4); meso-tetra-(4-sulfonatophenyl)-porphine dihydrochloride (H₂TPPS4); tetrakis (4-n-butylpyridiniumyl) porphyrin (TPyPH₂(N-Bu)); and tetra-N-octyl tetrakis pyridinium porphyrin (TPyPH₂(N-Oc)).

92. The method according to claim 70, wherein the porphyrin is protoporphyrin IX.

93. The method according to claim 70, wherein the porphyrin concentration in the fluid medium after the contacting step is from about 10⁻²M to about 10⁻⁵ M.

94. The method according to claim 70, wherein the porphyrin concentration in the fluid medium after the contacting step is from about 10⁻³ M to about 10⁻⁵ M

95. The method according to claim 70, wherein the contacting step comprises contacting the blood product with a solid matrix onto which a porphyrin has been absorbed.
